(19) United States
(12) Patent Application Publication
(10) Pub. No.: US 2025/0275910 A1
(43) Pub. Date: Sep. 4, 2025

(54) Cosmetic application of viniferine

(71) Applicant: Tomcat International Limited, London (GB)

(72) Inventors: Mathilde THOMAS, PARIS (FR); Bertrand THOMAS, PARIS (FR)

(21) Appl. No.: 18/294,258

(22) PCT Filed: Aug. 2, 2022
(86) PCT No.: PCT/EP2022/071615

§ 371 Date: Feb. 1, 2024

(30) Foreign Application Priority Data
Aug. 3, 2021 (FR) 21/08421

(51) Int. Cl. A61K 8/9789; A61Q 17/04; A61Q 19/02

(52) U.S. Cl. CPC A61K 8/9789; A61Q 17/04; A61Q 19/02; A61K 2800/522

(57) ABSTRACT

The invention concerns the cosmetic use of viniferine to prevent, reduce or erase damage caused by an exposure to blue light on the skin or the appendages.

### TECHNICAL FIELD

The present invention concerns the cosmetic use of viniferine to prevent, reduce or erase damage caused by blue light on the skin or appendages.

### BACKGROUND

The wavelengths of blue light are between 380 and 500 nm and are emitted by the sun but also and above all by artificial light sources, in particular by LED bulbs and all screens, omnipresent in daily life. Given the time of exposure to this light that this represents, numerous works have been initiated over the past ten years to assess the risks. These studies repeatedly demonstrate that blue light has harmful effects on the eyes and sleep, and it has recently been shown that it has deleterious consequences on the skin and the appendages. It seems to act on the skin by mechanisms different from those involved by UVB and UVA, but it causes stronger and more lasting hyperpigmentation [L. Duteil et al. Differences in visible light induced-pigmentation according to wavelength: a clinical and histological study in comparison with UVB exposure, Pigment Cell Melanoma Res. 2014 September;27(5):822-6]. The production of melanin by melanocytes is stimulated via activation of a protein, opsin-3, in response to its exposure to blue light [C. Regazzetti et al. Melanocytes Sense Blue Light and Regulate Pigmentation through Opsin-3, Journal of Investigative Dermatology (2018) Vol. 138, 171-178]. This damage is mainly observed on dark skin phototypes of type III and more, but it has also been demonstrated that blue light is involved in skin aging by altering the structure of keratinocytes and fibroblasts and by lowering collagen and elastin synthesis.

Various cosmetic compositions have been developed, some of which are commercially available, to protect the skin from the disadvantages of prolonged exposure to blue light. They comprise active ingredients capable of forming a barrier screen at wavelengths based on photo-protective molecules, associated with anti-aging, moisturizing, anti-oxidant, anti-radical agents, making it possible to limit the aforementioned harmful effects on the skin. It is clear that most do not target hyperpigmentation specifically caused by exposure to blue light.

### BRIEF SUMMARY

The invention aims to remedy this lack. According to the invention, it was accidentally discovered that viniferine had the power to overcome this draw back.

By viniferine according to the invention is meant a polymer of resveratrol whose monomeric units are selected from cis-resveratrol and trans-resveratrol, and in particular εviniferine and δ-viniferine which are dimers of resveratrol, and α-viniferine which is a cyclic trimer of resveratrol. In the present text and precisely in the experimental part, reference is made to ε-viniferine, and even more particularly to trans-ε-viniferine, but the invention is not restricted to the use of this dimer alone, it extends to resveratrol oligomers, having from 2 to 10, preferably from 2 to 4, resveratrol units (trans and/or cis).

The hydroxyl groups of resveratrol are very unstable in air and light, which is why it is relevant to resort to their conversion into a stable group, in particular into an ether group or an ester group. Thus, the invention includes the use of any aforementioned oligomer of which at least one hydroxyl group is in the form of an ether or ester group. Advantageously, the ether group to corresponds the formula —O—A and the ester group corresponds to the formula —O—CO—A, in which A represents a hydrocarbon radical preferably having from 1 to 28 carbon atoms, saturated or unsaturated, linear or branched, aliphatic, cyclic or alicyclic. According to a preferred variant, A is a radical of a fatty acid, saturated or unsaturated, and better still it is selected from the radicals of saturated or unsaturated fatty acids, at C16.

Thus, the invention concerns the cosmetic use of viniferine to prevent, reduce or erase damage caused by exposure to blue light on the skin or the appendages. It also concerns a cosmetic treatment method for preventing, reducing or erasing damage caused by exposure to blue light on the skin or appendages, said method comprising the application to the skin or appendages of a cosmetic composition comprising viniferine. As demonstrated in the examples, a cosmetic use or method according to the invention makes it possible in particular to prevent, reduce or erase the hyperpigmentation caused by an exposure to blue light.

The use and cosmetic treatment according to the invention can, respectively, meet the following characteristics, considered alone or in combination:
the viniferine is preferably selected from any of the resveratrol oligomers mentioned above; advantageously, it is ε-viniferine and even better trans-ε-viniferine;
the viniferine may include at least one hydroxyl group in the form of an ester or in the form of an ether, and in particular an ester or an ether as defined above. When one or more hydroxyl groups are in ester form, it is preferably a fatty acid ester, and better still a palmitic acid ester;
the viniferine can be used or present in the form of a single oligomer, in the form of a mixture of oligomers; whether in the form of a single oligomer or in the form of a mixture of oligomers, it can be combined with resveratrol, whether cis or trans, or even in a mixture.

The viniferine can be extracted from different plants, and in particular from the vine, of which the most common species Vitis vinifera is preferred. It can be obtained from different parts of the vine, for example fruits or parts of the fruit such as seeds or skin, but also from shoots or vine stalks. It can be extracted from fresh shoots or dry shoots.

According to a variant of the invention, viniferine is in the form of a plant extract containing it, and in particular an extract of vine shoots or stems. It is then combined with resveratrol and other polyphenols present in the vine shoots and stalks.

Such a plant extract is obtained by bringing the plant into contact with water and/or an organic solvent. In this way, the viniferine, as well as resveratrol and other monomers, as well as all other resveratrol oligomers which are present in the plant are solubilized in water and/or the organic solvent. The extraction can be carried out by subjecting the mixture of plant and water and/or organic solvent to a treatment such as microwaves, ultrasound, maceration-leaching or even supercritical fluids. Advantageously, the plant extract thus recovered can also be subjected to one or more additional extraction steps using an organic solvent (for example ethyl acetate or ethyl ether). Then, the plant extract can be washed and stored in freeze-dried form. When the plant extract is in freeze-dried form, it is a dry plant extract.

A viniferine composition intended for the cosmetic treatment of the invention advantageously comprises 0.001% to 1% by weight of viniferine relative to the weight of the composition.

In its application according to the invention, the viniferine can be combined with any other ingredient, in particular active ingredient, and for example any photoprotective agent making it possible to protect the skin and the appendages from blue light or with a view to a more broad in action, any agent that protects the skin and appendages from other harmful radiation such as UVA and UVB, but also any active ingredient that strengthens the skin's defenses against damage caused by blue light, or facilitating their repair. It will be advantageously associated with any excipient conventionally used in a cosmetic composition such as perfumes, preservatives, surfactants, texturizers. Such a composition may be in any dosage form normally used in cosmetics.

### BRIEF DESCRIPTION OF THE DRAWINGS

The invention is illustrated below in support of experimental tests demonstrating the effect of viniferine and the following figures.

FIG. 1 represents the melanin content expressed as a percentage of optical density at 405 nm of a culture of untreated HEM cells (NT) or treated with 16 μg/mL of the composition C1 (C1) or with 2 μg/mL of the composition C2 (C2), before an exposure to blue light at 412 nm or 450 nm under the conditions described below for the melanin dosage, relative to the optical density at 405 nm of a culture of untreated and non-irradiated HEM cells (TN for negative control).

FIG. 2 represents the melanin content expressed as a percentage of optical density at 405 nm of untreated human skin explants (NT) or treated with the composition C11 (C11) or with the composition C12 (C12), before an exposure to the blue light at 412 nm or 450 nm under the conditions described below for the melanin dosage, relative to the optical density at 405 nm of untreated and non-irradiated explant (TN for negative control).

### DETAILED DESCRIPTION AND EXAMPLES

In the following experimental part, the preventive effect of ε-viniferine (trans-ε-viniferine) at different concentrations, on the damage caused by blue light at two wavelengths (412 nm, characteristic of solar blue light and 450 nm, characteristic of blue light from screens) was evaluated by means of a test for determining the melanin content of cells treated with a composition of the invention. In order to determine the maximum concentrations to be tested, the toxicity of different concentrations of the compositions tested was first evaluated via the MTT test, a live cell counting test involving the MTT tetrazolium salt as a reagent and well known to the professional.

### Example 1: Efficacy of a Composition of the Invention Evaluated in a Test on Human Epidermal Melanocytes

tested ε-viniferine compositions

The following compositions of ε-viniferine were tested.

C1 is a plant extract, obtained from dry vine shoots, containing 10 to 30% trans-resveratrol (m/m) and 10 to 20% trans-ε-viniferin (m/m).

C2 is 95% purified trans-ε-viniferine.

### Cells

The tests were carried out on human epidermal melanocytes (HEM), which are cells that produce melanin and are therefore responsible for skin pigmentation. The HEM cells are cultured on a specific complete growth medium, at 37° C. and in an atmosphere of 5% CO₂.

### Melanin Content Test

The HEM cells were cultured at 37° C., in a 5% CO₂ atmosphere and treated with a determined concentration of the composition C1 or the composition C2.

After 24 hours of incubation at 37° C., in an atmosphere of 5% CO₂, the cells are exposed to blue light, 412 nm or 450 nm or kept in the dark (control) for 5 hours per day, for 3 days, representing a total irradiation of 120 J/cm².

24 hours after the last irradiation, the cells are washed, detached and centrifuged. In order to lyse the cells, the cell pellets are solubilized in a 1M NaOH solution at 80° C. for 1 hour, allowing the melanin to be solubilized.

For each test, the following were carried out:
a blank test in which the cells are not treated with one of the compositions C1 and C2 and are exposed to blue light under the same conditions as the tests;
a negative control (TN) in which the cells are not treated with one of the compositions C1 and C2 and are kept in the dark and therefore not exposed to blue light; and
a control in which the cells are treated with the composition C1 or the composition C2 and are kept in the dark and therefore not exposed to blue light.

The tests are carried out in triplicate.

### Evaluation of Test Results

The coloring intensity determined by the optical density measured at 405 nm is directly correlated to the melanin content.

The melanin content is calculated by relating the absorbance value to that of the negative control.

The percentage variation in melanin content compared to the blank test or the negative control is calculated according to the following equation:

% change in melanin content= [Abs₄₀₅ₑ−Abs₄₀₅ₜ]−[Abs₄₀₅ₜ−Abs_{405b}]×100

in which

Abs₄₀₅ₑ is the average value of the absorbance measured in each treatment condition,

Abs₄₀₅ₜ is the average absorbance value measured in each blank test

Abs_{405b} is the average absorbance value measured in the negative control.

### Results

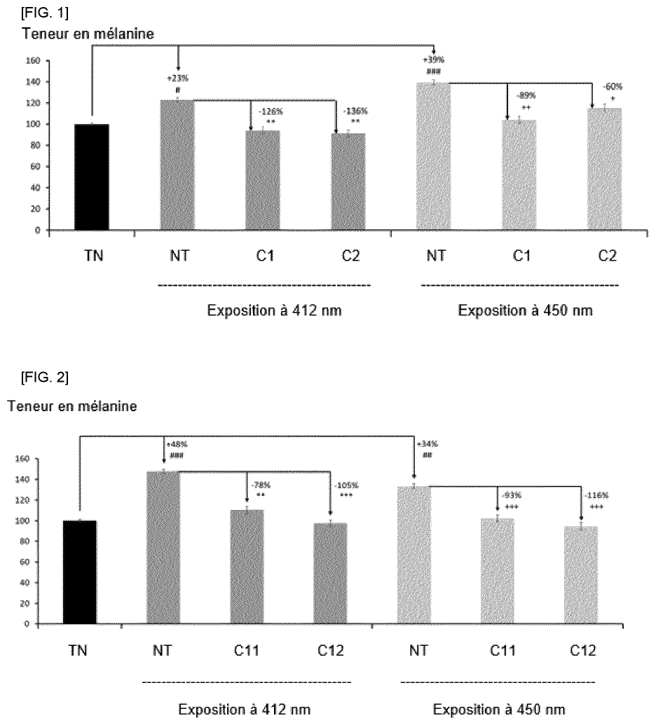

They are shown in FIG. 1. The data collected in this figure represent the mean±SEM of two experiments carried out in 6 to 18 replicates. The effect of the compositions on the pigmentation is calculated in relation to the induction of pigmentation between the untreated control and negative control (TN), according to the Mann-Whitney statistical test:
#p<0.05, ###p<0.001, versus TN
**p<0.01, versus NT for blue light at 450 nm
+p<0.05;++p<0.01, versus NT for blue light at 412 nm

It is first observed that an exposure of melanocytes to blue light causes a significant increase in melanin production, at 412 nm and 450 nm.

It is then seen that the treatment of melanocytes with ε-viniferine makes it possible to significantly reduce the production of melanin. For blue light at 450 nm, it is seen that the effectiveness of ε-viniferine is such that it makes it possible to lower the melanin content to the content of a culture of melanocytes not exposed to blue light.

### Example 2: Efficacy of a Composition of the Invention Evaluated in a Test on Human Skin Explants

tested Viniferine compositions

The following viniferine compositions were tested.

C11 and C12 are two compositions containing respectively 0.05% (m/m) and 0.1% (m/m) of viniferine (trans-ε-viniferine) in the form of an extract of dry vine shoots comprising viniferine, most of the hydroxyl groups of which have been esterified to palmitate. The formulation of the composition C11 is indicated in Table 1 and that of the composition C12 is indicated in Table 2 below where the constituents are named under their INCI name of the International Nomenclature of Cosmetic Ingredients.

TABLE 1

| INCI Name | % (m/m) |
|---|---|
| AQUA/WATER/EAU | QSP 100 |
| COCO-CAPRYLATE/CAPRATE | 19.991 |
| METHYLPROPANEDIOL | 1.640 |
| ARACHIDYL ALCOHOL | 1.620 |
| BEHENYL ALCOHOL | 0.840 |
| ARACHIDYL GLUCOSIDE | 0.540 |
| PALMITOYL GRAPEVINE | QSP VINIFERINE 0.050 |
| SHOOT EXTRACT | |
| CAPRYLYL GLYCOL | 0.300 |
| POLYACRYLATE CROSSPOLYMER-6 | 0.300 |
| POTASSIUM SORBATE | 0.100 |
| SODIUM BENZOATE | 0.100 |
| XANTHAN GUM | 0.100 |
| CITRIC ACID | 0.062 |
| PHENYLPROPANOL | 0.060 |
| TOCOPHEROL | 0.009 |

TABLE 2

| INCI Name | % (m/m) |
|---|---|
| AQUA/WATER/EAU | QSP 100 |
| COCO-CAPRYLATE/CAPRATE | 19.991 |
| METHYLPROPANEDIOL | 1.640 |
| ARACHIDYL ALCOHOL | 1.620 |
| BEHENYL ALCOHOL | 0.840 |
| PALMITOYL GRAPEVINE | QSP VINIFERINE 0.100 |
| SHOOT EXTRACT | |
| ARACHIDYL GLUCOSIDE | 0.540 |
| CAPRYLYL GLYCOL | 0.300 |
| POLYACRYLATE | 0.300 |
| CROSSPOLYMER-6 | |
| POTASSIUM SORBATE | 0.100 |
| SODIUM BENZOATE | 0.100 |
| XANTHAN GUM | 0.100 |
| CITRIC ACID | 0.062 |
| PHENYLPROPANOL | 0.060 |
| TOCOPHEROL | 0.009 |

### Explants

The tests were carried out on human skin explants obtained from plastic surgery. The biopsies are divided into explants of 8 mm diameter and kept for survival in complete medium. at 37° C. and in an atmosphere of 5% CO₂.

### Melanin Content Test

The skin explants are exposed to blue light, at 412 nm or 450 nm or kept in the dark (control) for 5 hours per day, for 3 days, representing a total irradiation of 120 J/cm². Before each exposure, the explants are treated overnight with the compositions C11 or C12.

24 hours after the last irradiation, the explants are collected and incubated in a 1 M NaOH solution at 80° C. for 3 hours, allowing the melanin to solubilize.

For each test, the following were carried out:
a blank test in which the explants are not treated and are exposed to blue light under the same conditions as the tests;
a negative control (TN) in which the explants are not treated and are kept in the dark and therefore not exposed to blue light; and
a control in which the cells are treated with the composition C1 or the composition C2 and are kept in the dark and therefore not exposed to blue light.

The tests are carried out in triplicate, on three explants.

### Evaluation of Test Results

The coloring intensity determined by the optical density measured at 405 nm is directly correlated to the melanin content.

The melanin content is calculated by relating the absorbance value to that of the negative control.

The percentage variation in melanin content compared to the blank test or the negative control is calculated according to the following equation:

% change in melanin content=[Abs₄₀₅ₑ−Abs₄₀₅ₜ]−[Abs₄₀₅ₜ−Abs_{405b}]×100

in which

Abs₄₀₅ₑ is the average value of the absorbance measured in each treatment condition,

Abs₄₀ₛₜ is the average absorbance value measured in each blank test

Abs_{405b} is the average absorbance value measured in the negative control.

### Results

They are shown in FIG. 2. The data collected in this figure represent the mean±SEM of the experiments carried out in triplicate, on 3 explants. The effect of the compositions on the pigmentation is calculated in relation to the induction of pigmentation between the untreated control and negative control (TN), according to the ANOVA statistical test:
##p<0.01. ###p<0.001, versus TN
**p<0.01, ***p<0.001, versus NT for blue light at 450 nm
+++p<0.001, versus NT for blue light at 412 nm

It is first observed that an exposure of explants to blue light causes a significant increase in melanin production, at 412 nm and 450 nm.

It is then seen that the treatment of the explants with C11 or C12 containing εviniferine makes it possible to significantly reduce the production of melanin. It is seen that the effectiveness of ε-viniferine is such that it makes it possible to lower the melanin content to the content of an explant not exposed to blue light.

### Example 3: Formulation of an Anti-Blue Light Serum

Table 3 below lists the constituents under their INCI name of an anti-blue light serum and their content in % by mass relative to the total mass of the serum. It contains viniferine (trans-ε-viniferine) in a use according to the invention.

TABLE 3

| INCI Name of constituents | % (m/m) |
|---|---|
| WATER | QSP 100 |
| BUTYLENE GLYCOL | 7.000000 |
| GLYCERIN | 5.000000 |
| COCO-CAPRYLATE/CAPRATE | 1.000000 |
| SQUALANE | 1.000000 |
| POLYGLYCERYL-3 DISTEARATE | 0.500000 |
| PALMITOYL GRAPEVINE SHOOT EXTRACT | 0.1 to 1 |
| BISABOLOL | 0.600000 |
| GLYCERYL STEARATE | 0.500000 |
| XANTHAN GUM | 0.200000 |
| PARFUM (FRAGRANCE) | QSP |
| PRESERVTIVE | QSP |
| MICROCRYSTALLINE CELLULOSE | 0.100000 |
| CITRIC ACID | QS pH = 5.5 |
| SODIUM PHYTATE | QSP |

Palmitoyl grapevine shoot extract is an extract of dried vine shoots comprising viniferine, the majority of the hydroxyl groups of which have been esterified into palmitate. The content indicated for this extract is equivalent to a viniferine content in this formulation falling in the range of 0.001 to 1% (m/m).

### Example 4: Formulation of an Anti-Blue Light Cream

Table 4 below lists the constituents, under their INCI name of the International Nomenclature of Cosmetic Ingredients, of an anti-blue light cream and their content in % by mass relative to the total mass of the serum. It contains viniferine in a use according to the invention.

TABLE 4

| Mixed INCI Name | % INCI |
|---|---|
| WATER | QSP 100 |
| BUTYLENE GLYCOL | 7.000000 |
| GLYCERIN | 4.000000 |
| CETEARYL ALCOHOL | 2.400000 |
| COCO-CAPRYLATE/CAPRATE | 1.000000 |
| GLYCERYL STEARATE SE | 1.000000 |
| BUTYROSPERMUM PARKII (SHEA) | 1.000000 |
| BUTTER EXTRACT | |
| CAPRYLIC/CAPRIC TRIGLYCERIDE | 1.000000 |
| VITIS VINIFERA (GRAPE) SEED OIL | 1.000000 |
| HYDROGENATED VEGETABLE OIL | 0.500000 |
| TRIHEPTANOIN | 0.200000 |
| PANTHENOL | 0.100000 |
| POTASSIUM CETYL PHOSPHATE | 0.100000 |
| SQUALANE | 0.100000 |
| GLYCOLIC ACID | QS PH = 5.5 |
| CETEARYL GLUCOSIDE | 0.500000 |
| BISABOLOL | 0.500000 |
| TOCOPHERYL ACETATE | 0.500000 |
| PARFUM (FRAGRANCE) | QSP |
| PRESERVATIVE | QSP |
| PALMITOYL GRAPEVINE SHOOT | 0.1 to 1 |
| EXTRACT | |
| ACACIA SENEGAL GUM | 0.250000 |
| XANTHAN GUM | 0.200000 |
| CAPRYLYL GLYCOL | 0.200000 |
| CASSIA ANGUSTIFOLIA SEED | 0.160000 |
| POLYSACCHARIDE | |
| PAPAIN | 0.007500 |
| TOCOPHEROL | 0.005000 |
| ALGIN | 0.002000 |

Palmitoyl grapevine shoot extract is an extract of dried vine shoots comprising viniferine, the majority of the hydroxyl groups of which have been esterified into palmitate. The content indicated for this extract is equivalent to a viniferine content in this formulation falling in the range of 0.001 to 1% (m/m).

1. A cosmetic comprising viniferine configured to protect, prevent, reduce or erase damage caused by an exposure to blue light on the skin or the appendages.

2. The cosmetic according to claim 1, wherein at least one hydroxyl group of viniferine is in the form of an ester or in the form of an ether.

3. The cosmetic according to claim 2, wherein at least one hydroxyl group of viniferine is in the form of a fatty acid ester, preferably a palmitic acid ester.

4. The cosmetic according to claim 1, wherein viniferine is associated with resveratrol and/or one of its oligomers.

5. The cosmetic according to claim 1, wherein the viniferine is in the form of a vine shoot extract.

6. A cosmetic treatment method for protecting, preventing, reducing or erasing damage caused by an exposure to blue light on the skin or the appendages, wherein it comprises the application to the skin or the appendages of a cosmetic composition comprising viniferine.

7. The method according to claim 6, wherein the viniferine is in the form of an ester or an ether, preferably in the form of a fatty acid ester such as an ester of palmitic acid.

8. The method according to claim 6, wherein the cosmetic composition comprises 0.001% to 1% by weight of viniferine relative to the weight of the composition.
